(19) United States
(12) Patent Application Publication
(10) Pub. No.: US 2024/0374297 A1
(43) Pub. Date: Nov. 14, 2024

(54) PUSH TYPE ANCHOR FOR CORTICAL BONE

(71) Applicant: AIRS INC., Daegu (KR)

(72) Inventors: Sanghyun JOUNG, Daegu (KR); Jungho CHO, Daegu (KR); Daehee RYU, Daegu (KR)

(21) Appl. No.: 18/783,051

(22) Filed: Jul. 24, 2024

Related U.S. Application Data
(63) Continuation of application No. PCT/KR2022/020467, filed on Dec. 15, 2022.

(30) Foreign Application Priority Data
Jan. 26, 2022 (KR) 10-2022-0011812

(51) Int. Cl. A61B 17/84

(52) U.S. Cl. CPC A61B 17/844

(57) ABSTRACT

Provided is a push type anchor fixed to one side cortical bone that is capable of being insertedly fixed to one side cortical bone by means of pushing, while being configured to minimize damage on a patient's body. The push type anchor fixed to one side cortical bone may include an anchor sleeve having a header located on the lower end portion thereof in such a way as to be inserted into the cortical bone, enlarged by means of an external force, and then fixed to the cortical bone and an anchor pin inserted into the anchor sleeve in such a way as to generate the external force for enlarging the header if pushed.

### CROSS-REFERENCE TO RELATED APPLICATION(S)

This application is a continuation application of international application No. PCT/KR2022/020467, filed on Dec. 15, 2022, and claims priority to Korean Patent Application No. 10-2022-0011812, filed on Jan. 26, 2022, with the Korean Intellectual Property Office, the disclosures of which are incorporated herein in their entirety by reference.

### TECHNICAL FIELD

The present invention relates to a push type anchor fixed to one side cortical bone, more specifically to a push type anchor fixed to one side cortical bone that is capable of being insertedly fixed to one side cortical bone by means of pushing, while being configured to minimize damage on a patient's body.

### BACKGROUND ART

Generally, spine surgery or fracture treatment is performed using a device insertedly fixed to a bone according to a surgery or treatment method. An example of the device is disclosed in Korean Patent No. 10-1178488 entitled ‘Device fixed to bone,’ and another example is disclosed in Korean Patent No. 10-1813525 entitled ‘Pedicle screw having four screw thread.’

The above-mentioned conventional devices fixed to the bones have screw threads formed on the outer peripheral surfaces thereof, and through the rotations of the screw threads, they are fixed to the bones. However, the fixed methods of the devices through the rotations of the screw threads may cause a patient's body such as surrounding nerves, soft tissues, and the like to be damaged and generate heat around the bones in the process of being inserted into or removed from the bones to increase surgery or treatment time.

### DISCLOSURE

### Technical Problem

Accordingly, the present invention has been made in view of the above-mentioned problems occurring in the related art, and it is an object of the present invention to provide a push type anchor fixed to one side cortical bone that is capable of preventing damage on a patient's body and generation of heat, while being fixed to the cortical bone, thereby reducing surgery or treatment time and being easy in grafting, removal, and correction.

### Technical Solution

To accomplish the above-mentioned objects, according to the present invention, a push type anchor fixed to one side cortical bone may include an anchor sleeve having a header located on the lower end portion thereof in such a way as to be inserted into the cortical bone, enlarged by means of an external force, and then fixed to the cortical bone and an anchor pin inserted into the anchor sleeve in such a way as to generate the external force for enlarging the header if pushed.

In this case, the header of the anchor sleeve may have a header body equally divided into a plurality of bodies by means of a plurality of incised portions in such a way as to be enlarged through the external force, and the anchor sleeve may have a hollow portion formed at the inside thereof in such a way as to insert the anchor pin thereinto, and the hollow portion is located on the lower end of the header in such a way as to have a smaller diameter than the anchor pin so that if a pin header of the anchor pin is inserted into the hollow portion, the header is enlarged.

Further, each incised portion may have an enlarged range enlarging hole formed on the end thereof in a longitudinal direction thereof in such a way as to enlarge the enlarged range of the header.

Furthermore, the hollow portion of the anchor sleeve may have a slant surface formed on the peripheral surface thereof in such a way as to have a wide top and narrow bottom diameter, and the pin header may have a slant surface formed on the peripheral surface thereof in such a way as to have a wide top and narrow bottom diameter and a slant angle smaller than a slant angle of the slant surface of the hollow portion of the anchor sleeve with respect to a vertical line, so that the slant surface of the pin header may move along the slant surface of the hollow portion of the anchor sleeve and thus enlarge the header.

Besides, the slant angle of the slant surface of the hollow portion of the anchor sleeve may be different from the slant angle of the slant surface of the pin header, and otherwise, the length of the anchor pin or the header may be adjusted to control the enlarged range of the header.

In addition, the header may be separately coupled to the anchor sleeve in such a way as to be replaceable.

Further, the push type anchor fixed to one side cortical bone may further include a first mechanism fastening part located on top of the anchor sleeve in such a way as to be fixed to a body of a dedicated applicator and a second mechanism fastening part located on top of the anchor pin in such a way as to be fixed to a handle rotatably located inside the body of the dedicated applicator, wherein the anchor sleeve and the anchor pin may be coupled to each other by means of screw threads formed thereon, and through the rotation of the handle of the dedicated applicator whose body is fixed to the first mechanism fastening part, the pushing for the anchor pin may be adjusted.

The push type anchor fixed to one side cortical bone may further include a shock absorbing member located on the outer peripheral surface of the header on the underside of the anchor sleeve in such a way as to absorb the shock generated between the anchor sleeve and the cortical bone.

Additionally, the header may have a patterned friction protrusion formed on the outer peripheral surface thereof and have one or more patterns selected from a vertical line and horizontal line lattice pattern, a diagonal line lattice pattern, a dot pattern, and a wavy line and straight line mixing pattern.

The push type anchor fixed to one side cortical bone may further include a belt insertion groove formed along the circumferential directions of the anchor sleeve and the second mechanism fastening part and a fixing belt inserted into the belt insertion groove in such a way as to allow both end portions thereof to be connected to each other so that the anchor sleeve and the second mechanism fastening part may be fixed by means of the fixing belt.

### Advantageous Effects

According to the present invention, the push type anchor fixed to one side cortical bone is capable of preventing heat from being generated because there is no rotation of the screw thread in the cortical bone, unlike the conventional technology wherein the screw thread rotates and is thus fixed to the cortical bone, so that the damage on the patient's body can be minimized to reduce the risk of the surgery and to help the patient recovered.

Further, the push type anchor fixed to one side cortical bone according to the present invention is fixed to one side cortical bone, and even after a metal nail has been inserted into a medullary cavity, therefore, the anchor rotates and deforms the metal nail accurately. Furthermore, the anchor increases the range of mechanisms connected thereto.

Furthermore, the effectiveness of the push type anchor fixed to one side cortical bone according to the present invention is not limited as mentioned above, and it should be understood to those skilled in the art that the effectiveness of the invention may include another effectiveness as not mentioned above from the detailed description of the present invention.

### BRIEF DESCRIPTION OF DRAWINGS

FIG. 1 is a perspective view showing a push type anchor fixed to one side cortical bone according to the present invention.

FIG. 2 is an enlarged perspective view showing the push type anchor fixed to one side cortical bone according to the present invention.

FIGS. 3A to 3C are perspective views showing various shapes of a header of the push type anchor fixed to one side cortical bone of FIG. 1.

FIGS. 4A and 4B are perspective views showing various shapes of enlarged range enlarging holes of FIG. 3.

FIGS. 5A and 5B are sectional views showing the use states of the push type anchor fixed to one side cortical bone of FIG. 1.

FIGS. 6A and 6B are enlarged sectional views showing the regions close to the cortical bone in FIG. 5.

FIG. 7 is a perspective view showing a dedicated applicator only for the push type anchor fixed to one side cortical bone according to the present invention.

FIG. 8 is a sectional view showing the dedicated applicator of FIG. 7.

FIG. 9 is a perspective view showing a state where the push type anchor fixed to one side cortical bone according to the present invention is mounted onto the dedicated applicator of FIG. 7.

FIG. 10 is a sectional view showing the state of FIG. 9.

FIG. 11 is a sectional view showing a configuration and operation of a shock absorbing member provided for the push type anchor fixed to one side cortical bone according to the present invention.

FIGS. 12A to 12D are perspective views showing various examples of patterned friction protrusions provided for the push type anchor fixed to one side cortical bone according to the present invention.

FIGS. 13A and 13B are perspective and side views showing an example of a friction improving screw thread provided for the push type anchor fixed to one side cortical bone according to the present invention.

FIG. 14 is a sectional view showing a configuration and operation of a fixing belt provided for the push type anchor fixed to one side cortical bone according to the present invention.

### BEST MODE FOR INVENTION

Hereinafter, specific exemplary embodiments of the present invention are illustrated in the drawings and described in detail in the detailed description. However, this does not limit the invention within specific embodiments, and it should be understood that the invention covers all the modifications, equivalents, and replacements within the idea and technical scope of the invention.

Terms, such as the first, the second, and the like, may be used to describe various elements, but the elements should not be restricted by the terms. The terms are used to only distinguish one element from the other element.

In the description, the same reference numerals will be used to describe the same components.

An expression referencing a singular value additionally refers to a corresponding expression of the plural number, unless explicitly limited otherwise by the context. In this application, terms, such as “comprise,” “include,” or “have,” are intended to designate those characteristics, numbers, steps, operations, elements, or parts which are described in the specification, or any combination of them that exist, and it should be understood that they do not preclude the possibility of the existence or possible addition of one or more additional characteristics, numbers, steps, operations, elements, or parts, or combinations thereof.

Hereinafter, an explanation of a push type anchor fixed to one side cortical bone according to the present invention will be given in detail with reference to the attached drawings.

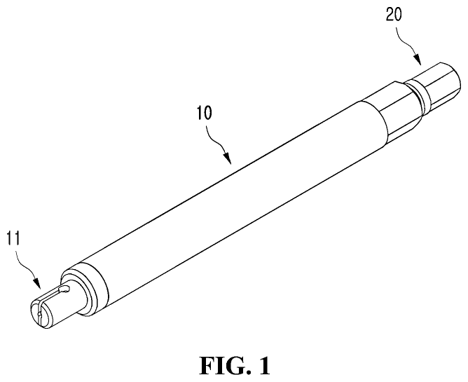

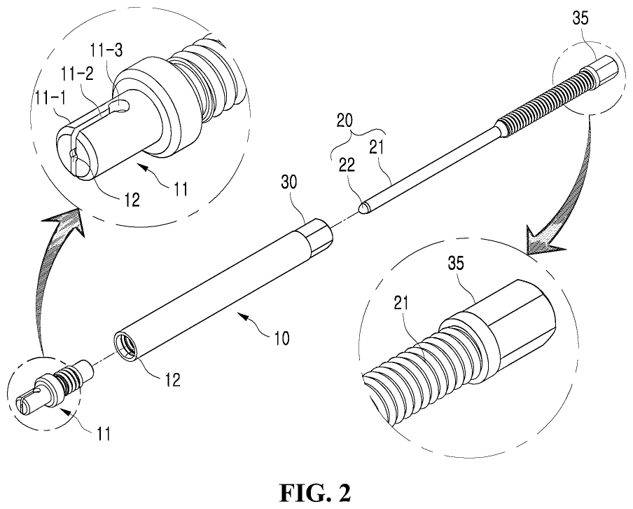

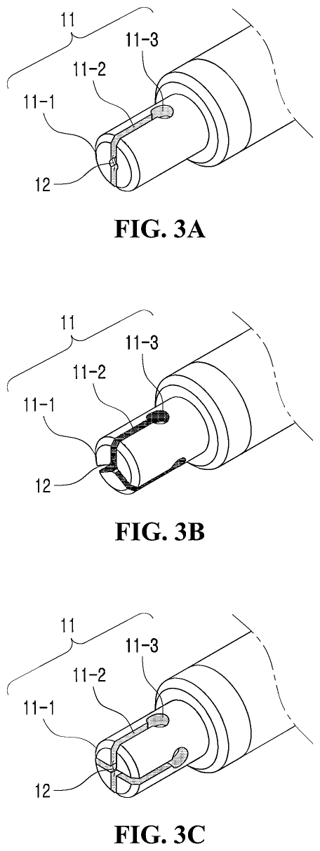

FIG. 1 is a perspective view showing a push type anchor fixed to one side cortical bone according to the present invention, FIG. 2 is an enlarged perspective view showing the push type anchor fixed to one side cortical bone according to the present invention, and FIGS. 3A to 3C are perspective views showing various shapes of a header of the push type anchor fixed to one side cortical bone of FIG. 1.

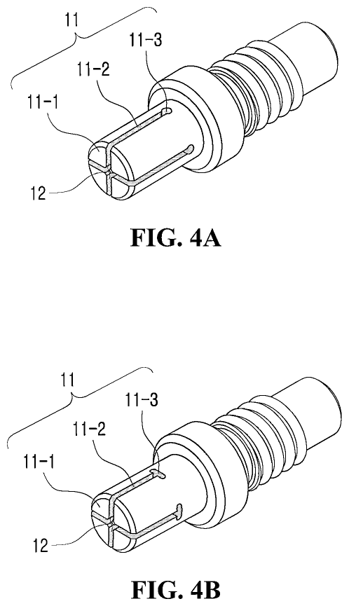

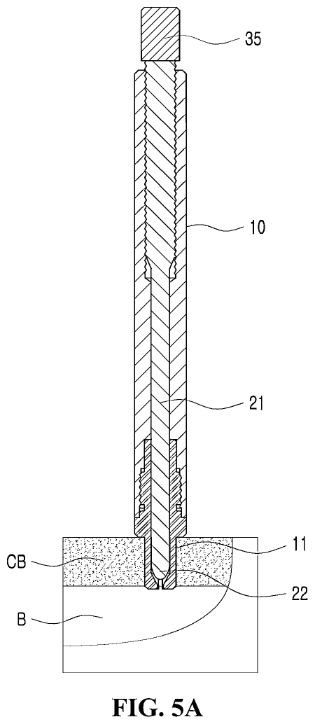

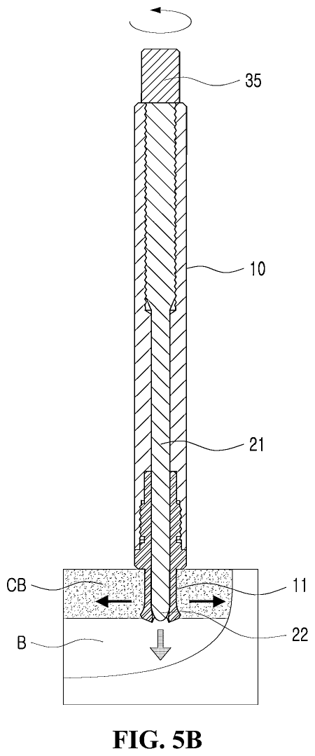

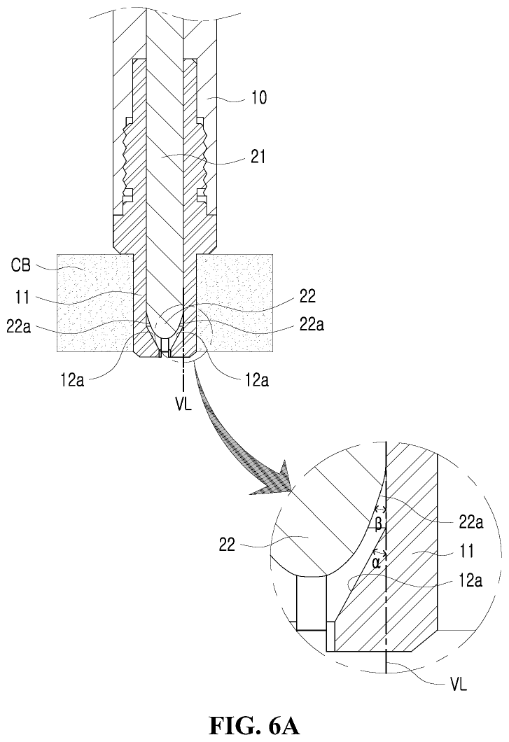

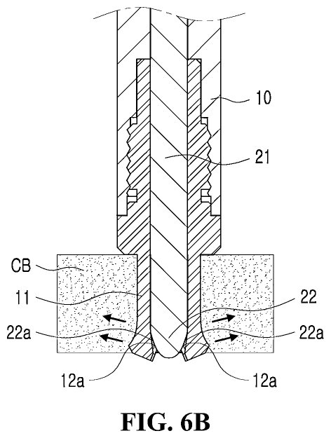

Further, FIGS. 4A and 4B are perspective views showing various shapes of enlarged range enlarging holes of FIG. 3, FIGS. 5A and 5B are sectional views showing the use states of the push type anchor fixed to one side cortical bone of FIG. 1, and FIGS. 6A and 6B are enlarged sectional views showing the regions close to the cortical bone in FIG. 5.

Referring to FIGS. 1 to 6B, the present invention relates to an anchor insertedly fixed to one side cortical bone CB for fracture surgery, especially minimal invasive fracture surgery, and the anchor includes an anchor sleeve 10 and an anchor pin 20 so that as the anchor pin 20 is pushedly inserted into the anchor sleeve 10, it is fixed to the inside of the cortical bone CB located on the outer layer of bone B.

In this case, the anchor sleeve 10 has a header 11 located on the lower end portion thereof in such a way as to be inserted into the cortical bone CB, enlarged by means of an external force, and then fixed to the cortical bone CB, and if the anchor pin 20 is inserted into the anchor sleeve 10 and then pushed, it generates the external force with which the header 11 is enlarged.

Under the above-mentioned configuration, the anchor according to the present invention is more easily fixed to and separated from the cortical bone CB and more securely minimizes the bone damage of a patient when compared with a conventional configuration wherein a screw thread rotates and is fixedly inserted into the bone.

There are various configurations for enlarging and fixing the header 11 of the anchor sleeve 10 inserted into the cortical bone CB by means of the anchor pin 20, but to achieve easy and simple use and to minimize the damage to the patient's bone, the anchor according to the present invention includes the header 11 having a header body 11-1 and incised portions 11-2 and the anchor pin 20 having a pin body 21 and a pin header 22.

In detail, the header body 11-1 is not restricted in shape, but it is desirably cylindrical and located on the lower end portion of the anchor sleeve 10 in such a way as to have a smaller diameter than the anchor sleeve 10. The incised portions 11-2 are incised in a direction from the center of the underside of the header body 11-1 toward the upper end portion of the header body 11-1. In this case, the incised portions 11-2 are formed in directions facing each other in such a way as to equally divide the header body 11-1 into a plurality of bodies.

The pin body 21 of the anchor pin 20 is not restricted in shape, but it is desirably cylindrical in such a way as to pass through the anchor sleeve 10 and be thus inserted into the anchor sleeve 10. To do this, the anchor sleeve 10 has a hollow portion 12 extending from top thereof to the underside of the header body 11-1 in such a way as to insert the anchor pin 20 thereinto. That is, the center of the plurality of incised portions 11-2 is connected to the hollow portion 12.

The pin header 22 is located on the underside of the pin body 21. In this case, the pin header 22 has a larger area than the hollow portion 12 of the anchor sleeve 10 that is located on the lower end of the header body 11-1. That is, the hollow portion 12 of the anchor sleeve 10, which is located on the lower end of the header body 11-1, is formed in a smaller area than the pin header 22, so that when the pin header 22 passes through the hollow portion 12, it is resistant to the hollow portion 12 to cause the header 11 to be enlarged and deformed.

According to the present invention, in this case, the header body 11-1 is equally divided by means of the incised portions 11-2, thereby being decreased in stiffness, and if the pin header 22 as an external force application body is inserted, the header body 11-1 is easily enlarged and deformed due to the stiffness decreased through the incised portions 11-1.

If the pin header 22 is inserted into the anchor sleeve 10 in a direction from top of the anchor sleeve 10 toward the bottom thereof by means of a tool or a surgeon's force, the pin header 22 moves downward along the hollow portion 12 of the anchor sleeve 10, and next, if the pin header 22 is located on the lower end of the narrow header body 11-1, the header body 11-1 can be enlarged. In this case, even if the pin header 22 is located on the lower end of the header body 11-1, desirably, the pin body 21 has a longer length than the anchor sleeve 10 in such a way as to allow the upper end portion thereof to protrude from top of the anchor sleeve 10.

Further, each incised portion 11-2 has an enlarged range enlarging hole 11-3 formed on the end thereof in a longitudinal direction thereof, that is, on top end thereof. The enlarged range enlarging hole 11-3 serves to enlarge the enlarged range formed only by means of the incised portion 11-2, and as shown in FIGS. 3A to 3C, the enlarged range enlarging holes 11-3 have circular holes having larger diameters than the incised portions 11-2. As shown in FIG. 4A, further, the enlarged range enlarging holes 11-3 have circular holes having relatively small diameters, and otherwise, as shown in FIG. 4B, the enlarged range enlarging holes 11-3 have long holes extending laterally from the incised portions 11-2. As a result, the enlarged range of the header 11 can be enlarged.

In the case of the hollow portion of the anchor sleeve (hereinafter, referred to as ‘anchor sleeve hollow portion’), further, the hollow portion 12 located on the lower side of the header 11 has a slant surface 12a formed on the peripheral surface thereof in such a way as to have a wide top and narrow bottom diameter, and the pin header 22 has a slant surface 22a formed on the peripheral surface thereof in such a way as to have a wide top and narrow bottom diameter, so that if the pin body 21 is pushed, the pin header 22 is inserted into the hollow portion 12 having the wide top and narrow bottom diameter of the anchor sleeve 10.

In this case, a slant angle β of the slant surface of the pin header 22 is smaller than a slant angle α of the slant surface of the anchor sleeve hollow portion 12 with respect to a vertical line VL, so that the slant surface 22a of the pin header 22 moves along the slant surface 12a of the anchor sleeve hollow portion 12 and thus enlarges the header 11.

If the slant angle α of the slant surface of the anchor sleeve hollow portion 12 is different from the slant angle β of the slant surface of the pin header 22 under the above-mentioned principle, the enlarged range of the header 11 is adjustable.

For example, if there is a small difference between the slant angle α of the slant surface of the anchor sleeve hollow portion 12 and the slant angle β of the slant surface of the pin header 22, the enlarged range of the header 11 is relatively large, and contrarily, if there is a big difference between the slant angle α of the slant surface of the anchor sleeve hollow portion 12 and the slant angle β of the slant surface of the pin header 22, the enlarged range of the header 11 is relatively small.

In addition to the difference between the slant angle α of the slant surface of the anchor sleeve hollow portion 12 and the slant angle β of the slant surface of the pin header 22, according to the present invention, the enlarged range of the header 11 can be controlled by means of the adjustment of the length of the anchor pin 20 or the header 11. In this case, if the length of the anchor pin 20 or the header 11 is adjusted, the degree of insertion of the anchor pin 20 with respect to the length of the header 11 is different from the degree of insertion thereof under the same conditions except for the adjustment of the length of the anchor pin 20 or the header 11, thereby making it possible to control the enlarged range of the header 11.

The adjustment in the enlarged range of the header 11 has a direct relation with the fixed force to the cortical bone CB, and therefore, it is useful in setting the fixed force to the cortical bone CB.

Even if not shown in the drawings, the header 11 is separately coupled to the anchor sleeve 10 in such a way as to be replaceable, thereby ensuring sanitary use.

In this case, screw thread coupling is performed between the header body 11-1, and the anchor sleeve 10 to allow the header 11 to be separately coupled to the anchor sleeve 10, and otherwise, a given fixing device may be provided therebetween.

Further, the anchor pin 20 is slidably inserted into the anchor sleeve 10 by means of the tool or the surgeon's force, but as shown in the drawings, desirably, the anchor sleeve 10 and the anchor pin 20 are coupled to each other by means of screw threads, so that the anchor pin 20 rotates and is thus inserted into the anchor sleeve 10. If the anchor pin 20 rotates and is thus inserted into the anchor sleeve 10, it is easy for the anchor pin 20 to be minutely adjusted and fixed in position.

In the case where the anchor pin 20 is slidably inserted into the anchor sleeve 10, contrarily, a fixing part for fixing the anchor pin 20 in position is additionally provided.

Further, the push type anchor fixed to one side cortical bone according to the present invention includes a first mechanism fastening part 30 and a second mechanism fastening part 35, thereby ensuring minute adjustment of the anchor pin 20 and many conveniences in the adjustment.

The first mechanism fastening part 30 is provided to the shape of a polygon on top of the anchor sleeve 10, and the second mechanism fastening part 35 is provided to the shape of a polygon on the portion exposed to the outside from top of the anchor sleeve 10 when the anchor pin 20 is inserted into the anchor sleeve 10. In this case, the polygon is desirably hexagon, but it may not be limited necessarily thereto.

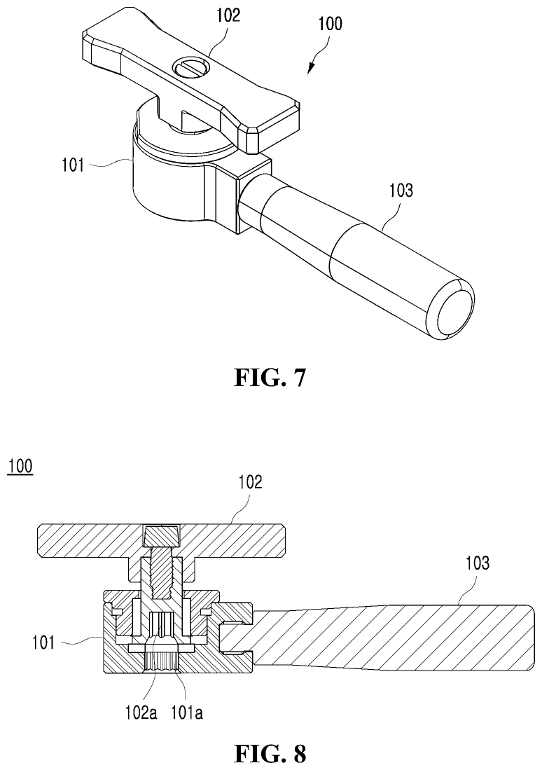

The first mechanism fastening part 30 and the second mechanism fastening part 35, which have the shapes of polygons, are fixed to a dedicated applicator 100, as shown in FIGS. 7 to 10 for mechanically adjusting the push type anchor fixed to one side cortical bone according to the present invention. In detail, a body 101 of the dedicated applicator 100 is fixed to the first mechanism fastening part 30, and a handle 102 of the dedicated applicator 100 is fixed to the second mechanism fastening part 35.

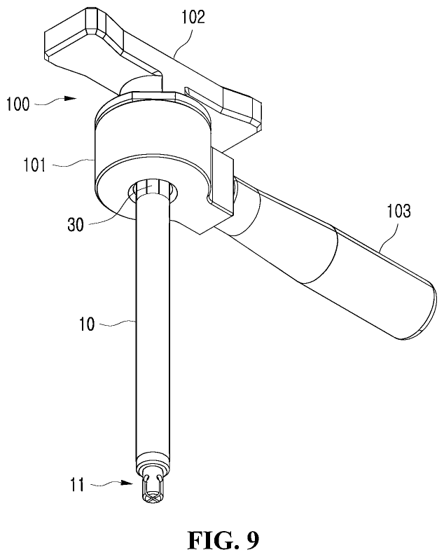

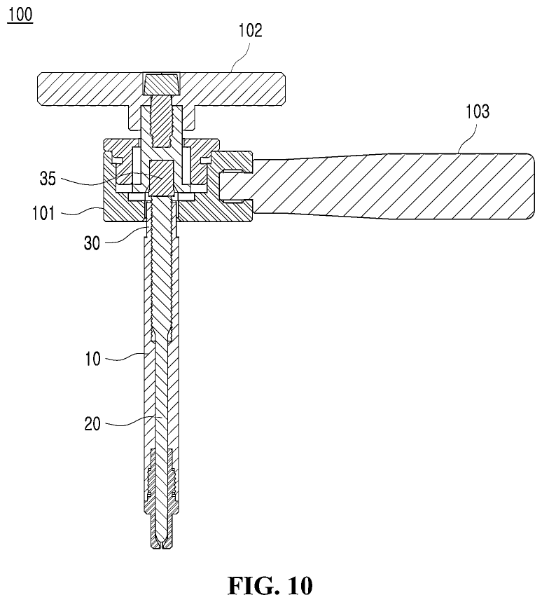

FIG. 7 is a perspective view showing a dedicated applicator for the push type anchor fixed to one side cortical bone according to the present invention, FIG. 8 is a sectional view showing the dedicated applicator of FIG. 7, FIG. 9 is a perspective view showing a state where the push type anchor fixed to one side cortical bone according to the present invention is mounted onto the dedicated applicator of FIG. 7, and FIG. 10 is a sectional view showing the state of FIG. 9.

Referring to FIGS. 7 to 10, the dedicated applicator 100 is configured to have the body 101 and the handle 102, and to improve the manipulation and grasping for the body 101, further, the dedicated applicator 100 has a grasping part 103 extending from the body 101.

In detail, the body 101 has a sleeve fastening groove 101a formed on the inside of the bottom thereof, while having the corresponding shape to the surface of the first mechanism fastening part 30, in such a way as to fix the first mechanism fastening part 30 thereto, and the handle 102 has a pin fastening groove 102a formed on the inside of the bottom thereof, while having the corresponding shape to the surface of the second mechanism fastening part 35, in such a way as to fix the second mechanism fastening part 35 thereto.

In this case, the handle 102 rotates inside the body 101 as the bottom on which the pin fastening groove 102a is formed is inserted into the body 101. In this case, screw threads (not shown) are formed between the body 101 and the handle 102, and if the handle 102 rotates inside the body 101, therefore, it moves up or down.

Further, if the anchor sleeve 10 and the anchor pin 20 are coupled to each other through the screw threads to allow the anchor pin 20 to be inserted into the anchor sleeve 10, a rubber bushing (not shown) is disposed between the underside of the handle 102 and the body 101, so that the handle 102 rotates in place on top of the rubber bushing to allow only the anchor pin 20 to move down.

In detail, the screw thread or rubber bushing is located inside the body 101 to allow the handle 102 to move up and down together with the anchor pin 20 or to allow only the anchor pin 20 to move up and down.

Further, the handle 102 is not restricted in shape, but desirably, it has the shape of “T” capable of being easily grasped or manipulated. The grasping part 103 is detachably mounted on the body 101.

As a result, the anchor pin 20 rotatably moves along the screw thread in the anchor sleeve 10 through the rotation of the handle 102 of the dedicated applicator 100, whose body 101 is fixed to the anchor sleeve 10 and thus moves upward or downward, and while being moved, if the pin header 22 located on the underside of the anchor pin 20 is positioned on the lower side of the header 11, the header 11 is enlarged. Accordingly, the anchor pin 20 is adjustable more precisely and conveniently by means of the surgeon.

Under the above-mentioned configuration, the push type anchor fixed to one side cortical bone according to the present invention is capable of preventing heat from being generated from the cortical bone because there is no rotation of the screw thread in the cortical bone, unlike the conventional technology wherein the screw thread rotates and is thus fixed to the cortical bone, so that the damage on the patient's body can be minimized to reduce the risk of the surgery and to help the patient recovered.

Further, the push type anchor fixed to one side cortical bone according to the present invention is fixed to one side cortical bone CB, and even after a metal nail has been inserted into a medullary cavity, therefore, the anchor rotates, deforms, and corrects the metal nail in position accurately. Furthermore, the anchor increases the selection range of mechanisms connected thereto.

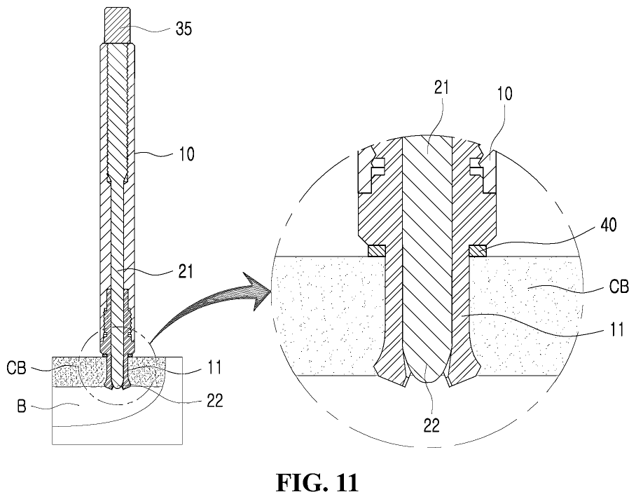

Referring to FIGS. 11 to 14, the push type anchor fixed to one side cortical bone according to the present invention may further include a shock absorbing member 40, a patterned friction protrusion 50, or a fixing belt 60.

FIG. 11 is a sectional view showing a configuration and operation of the shock absorbing member provided for the push type anchor fixed to one side cortical bone according to the present invention.

Referring to FIG. 11, the shock absorbing member 40 is located on the outer peripheral surface of the header 11 on the underside of the anchor sleeve 10. That is, the shock absorbing member 40 is fitted to the header 11 in such a way as to be located on the underside of the anchor sleeve 10. In this case, the shock absorbing member 40 is made of rubber or silicone and serves to absorb shocks generated between surfaces, and when surgery is performed with the anchor of the present invention, accordingly, the shock absorbing member 40 absorbs the shock generated between the anchor sleeve 10 and the cortical bone CB to prevent the cortical bone CB from being damaged due to the friction against the anchor sleeve 10.

Further, the shock absorbing member 40 is fitted to a groove (not shown) formed on the header 11 and thus does not escape from the header 11 easily, and the outer peripheral surface of the shock absorbing member 40 has the same diameter as the anchor sleeve 10, thereby preventing the cortical bone CB from being damaged due to the underside of the anchor sleeve 10.

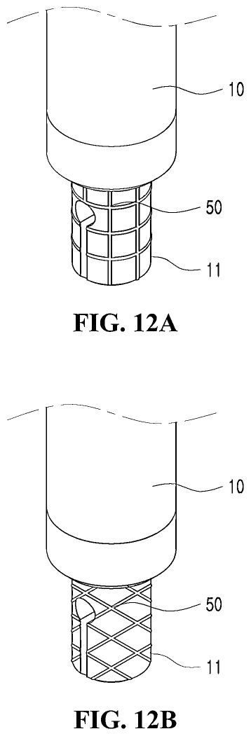

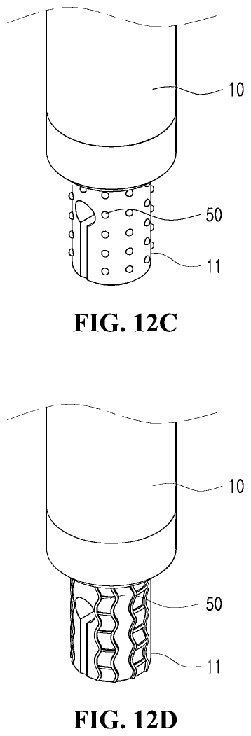

FIGS. 12A to 12D are perspective views showing various examples of patterned friction protrusions provided for the push type anchor fixed to one side cortical bone according to the present invention.

Referring to FIGS. 12A to 12D, the header 11 has the patterned friction protrusion 50 formed on the outer peripheral surface thereof. In detail, as shown in FIG. 12A, the patterned friction protrusion 50 is formed to have a vertical line and horizontal line lattice pattern in which vertical lines and horizontal lines cross each other, and as shown in FIG. 12B, the patterned friction protrusion 50 is formed to have a diagonal line lattice pattern in which diagonal lines having different directions cross each other.

Further, as shown in FIG. 12C, the patterned friction protrusion 50 is formed to have a dot pattern in which only dot protrusions are arranged, and as shown in FIG. 12D, the patterned friction protrusion 50 is formed to have a wavy line and straight line mixing pattern in which wavy lines and straight lines are mixed.

In the case of the wavy line and straight line mixing pattern, as shown in FIG. 12D, the wavy lines are formed in upward and downward directions and the straight lines are formed horizontally between the neighboring wavy lines, but without being limited thereto, the wavy line and straight line mixing pattern may be freely changed. For example, the wavy lines are formed laterally on the outer peripheral surface of the header 11, while the straight lines are formed vertically between the neighboring wavy lines.

The above-mentioned patterned friction protrusions 50 may be mixed with one another or formed individually, and if the header 11 is enlarged by means of the anchor pin 20, the patterned friction protrusion 50 serves to improve the fixed force to the cortical bone CB. In this case, shear forces are formed in different directions according to the pattern of the patterned friction protrusion 50, thereby enhancing the fixed force to the cortical bone CB.

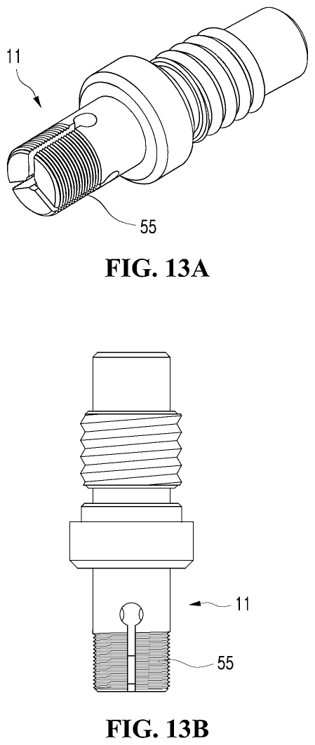

Further, as shown in FIG. 13, the header 11 has a friction improvement screw thread 55 formed on the outer peripheral surface thereof. FIGS. 13A and 13B are perspectives and side views showing an example of the friction improving screw thread provided for the push type anchor fixed to one side cortical bone according to the present invention.

Referring to FIGS. 13A and 13B, the friction improvement screw thread 55 serves to improve the fixed force to the cortical bone CB when the header 11 is enlarged, which is different from the conventional screw thread inserted by rotating into a bone. As shown, the friction improvement screw thread 55 is desirably formed only on a given height of the header 11 up to which the header 11 is enlarged, but without being limited necessarily thereto, even if not shown, the friction improvement screw thread 55 may be formed on the entire surface of the header 11.

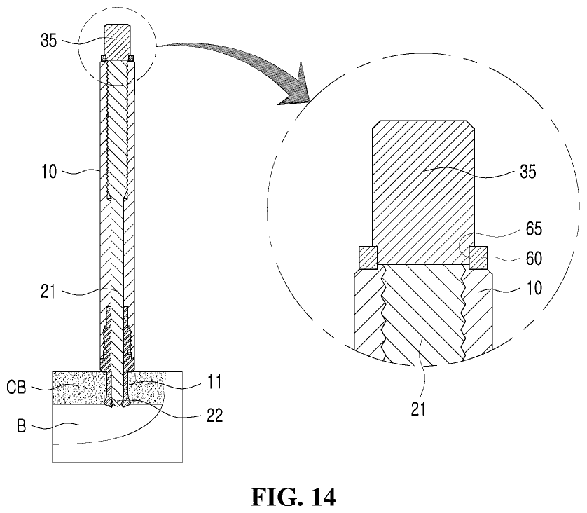

FIG. 14 is a sectional view showing a configuration and operation of a fixing belt provided for the push type anchor fixed to one side cortical bone according to the present invention.

Referring to FIG. 14, a belt insertion groove 65 is formed along the circumferential directions of the anchor sleeve 10 and the second mechanism fastening part 35. Further, a fixing belt 60 is inserted into the belt insertion groove 65 in such a way as to allow both end portions thereof to be connected to each other. When both end portions of the fixing belt 60 are connected to each other, the belt insertion groove 65 formed over the anchor sleeve and the second mechanism fastening part 35 is fastened, and further, the screw threads between the anchor sleeve 10 and the anchor pin 20 are fixedly fastened to each other, so that double connection can be performed.

As a result, the fixing belt 60 located inside the belt insertion groove 65 prevents the second mechanism fastening part 35 from separately moving upward from the anchor sleeve 10 due to the resistance of the cortical bone CB or bone B, so that the second mechanism fastening part 35 is more accurately and easily adjustable and the fixed force to the cortical bone is improved.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings. It is, therefore, intended that the scope of the invention be limited not by this detailed description but rather by the claims appended hereto.

### EXPLANATIONS OF REFERENCE NUMERALS

10: Anchor sleeve
11: Header
11-1: Header body
11-2: Incised portion
11-3: Enlarged range enlarging hole
12: Hollow portion
12a: Slant surface
20: Anchor pin
21: Pin body
22: Pin header
22a: Slant surface
30: First mechanism fastening part
35: Second mechanism fastening part
40: Shock absorbing member
50: Patterned friction protrusion
55: Friction improving screw thread
60: Fixing belt
65: Belt insertion groove
100: Dedicated applicator
101: Body
101a: Sleeve fastening groove
102: Handle
102a: Pin fastening groove
103: Grasping part
VL: Vertical line
CB: Cortical bone
B: Bone

1. An anchor for cortical bone comprising:
an anchor sleeve having a header located on a proximal end portion configured to be inserted into the cortical bone, enlarged by an external force, and fixed to the cortical bone; and
an anchor pin inserted into the anchor sleeve configured to transmit the external force for enlarging the header if the anchor pin is pushed to the cortical bone.

2. The anchor according to claim 1, wherein the header of the anchor sleeve has a header body proportionally divided into a plurality of bodies with a plurality of incised portions configured to enlarge through the external force,
wherein the anchor sleeve has a hollow portion configured to insert the anchor pin thereinto, the hollow portion being located on the proximal end of the header having a smaller diameter than the anchor pin wherein the header enlarges when a pin header of the anchor pin is inserted into the hollow portion.

3. The anchor according to claim 2, wherein each of the plurality of the incised portion has an enlarged range enlarging hole formed on the end thereof in a longitudinal direction configured to enlarge the enlarged range of the header.

4. The anchor according to claim 2, wherein the hollow portion of the anchor sleeve has a through-hole, and a first inner diameter thereof is greater than a second inner diameter located at the end of the through-hole,
the pin header has a slant surface on the peripheral surface extending between the first inner diameter and the second inner diameter,
wherein the slant surface is tapered in a longitudinal direction of the pin header and a slant angle of the slant surface of the pin header is smaller than a slant angle of the slant surface of the hollow portion of the anchor sleeve, and
wherein the slant surface of the pin header is configured to move along the slant surface of the hollow portion of the anchor sleeve which enlarges the header.

5. The anchor according to claim 4, wherein the slant angle of the slant surface of the hollow portion of the anchor sleeve is different from the slant angle of the slant surface of the pin header,
wherein the length of the anchor pin or the header is adjusted to control the enlarged range of the header.

6. The anchor according to claim 1, wherein the header is detachably attached to the anchor sleeve configured to be replaced.

7. The anchor according to claim 1, further comprising a first mechanism fastening part located on top of the anchor sleeve configured to be fixed to a body of a dedicated applicator; and
a second mechanism fastening part located on top of the anchor pin configured to be coupled to a handle rotatably located inside the body of the dedicated applicator,
wherein the anchor sleeve and the anchor pin are coupled with a plurality of screw threads thereon, and through a rotation of the handle of the dedicated applicator wherein the body is fixed to the first mechanism fastening part.

8. The anchor according to claim 1, further comprising a shock absorber located on the outer peripheral surface of the header between the anchor sleeve and the cortical bone,
wherein the shock absorber is configured to absorb the shock transmitted from the anchor sleeve to the cortical bone.

9. The anchor according to claim 1, wherein the header has a patterned friction protrusion on the outer peripheral surface wherein at least one pattern is selected from a vertical line and horizontal line lattice pattern, a diagonal line lattice pattern, a dot pattern, and a wavy line and straight-line mixing pattern.

10. The anchor according to claim 7, further comprising a belt insertion groove along the circumferential surface of the anchor sleeve and the second mechanism fastening part; and
a fixing belt inserted into the belt insertion groove configured to allow the anchor sleeve and the second mechanism fastening part to be connected to each other wherein the anchor sleeve and the second mechanism fastening part are coupled with the fixing belt.
